(19) United States
(12) Patent Application Publication
(10) Pub. No.: US 2022/0160602 A1
(43) Pub. Date: May 26, 2022

(54) LOW-WATER AGENTS FOR THE OXIDATIVE COLORING OF KERATIN FIBERS WITH PERBORATE, MICROBIAL GUM AND ANIONIC CELLULOSE

(71) Applicant: Henkel AG & Co. KGaA, Duesseldorf (DE)

(72) Inventors: Udo ERKENS, Willich (DE); Torsten LECHNER, Langenfeld (DE)

(73) Assignee: Henkel AG & Co. KGaA, Duesseldorf (DE)

(21) Appl. No.: 17/533,512

(22) Filed: Nov. 23, 2021

(30) Foreign Application Priority Data
Nov. 24, 2020 (DE) 102020214720.5

(51) Int. Cl. A61K 8/41; A61Q 5/10; A61K 8/73; A61K 8/19

(52) U.S. Cl. CPC A61K 8/411; A61K 8/19; A61K 8/731; A61Q 5/10

(57) ABSTRACT

The object of the present disclosure is an agent for the oxidative coloring of keratinous fibers, in particular human hair, comprising

(a) at least one perborate,
(b) at least one oxidation dye precursor,
(c) at least one microbial gum,
(d) at least one anionic cellulose, and
(e) less than 10% by weight of water, the percentage by weight being expressed in relation to the total weight of the product.

Another object is a process for the oxidative coloring of hair, in which a corresponding agent is used.

### CROSS-REFERENCE TO RELATED APPLICATION

This application claims priority to German Patent Application No. 102020214720.5, filed Nov. 24, 2020, which is incorporated herein by reference in its entirety.

### TECHNICAL FIELD

The present disclosure is in the field of cosmetics and relates to agents for the oxidative coloring of keratinous fibers, in particular human hair, comprising at least one perborate, at least one oxidation dye precursor, at least one microbial gum and at least one anionic cellulose. The agents are exemplified by a water content of not more than 10% by weight and are preferably prepared in the form of a coloring powder or paste.

Furthermore, the present disclosure relates to a process for the oxidative coloring of keratinous fibers, in which a ready-to-use agent is prepared from the agent by mixing it with water, and the latter is then applied to the keratinous fibers.

### BACKGROUND

Changing the color of keratin fibers, especially hair, is an important area of modem cosmetics. As a result, the appearance of the hair can be adapted both to current fashion trends and to the individual wishes of the individual. The expert knows different possibilities for changing the hair color.

The hair color can be changed temporarily by using direct dye. Here, already fully formed dyes from the dye diffuse into the hair fiber. The dyeing with direct dyes is associated with little damage to the hair, but a disadvantage is the short shelf life and the quick washability of the dyeing's obtained with direct dyes.

If the consumer desires a long-lasting color result or a shade that is lighter than his original hair color, oxidative color change agents are therefore usually used. For permanent, intensive dyeing with corresponding fastness properties, so-called oxidation dyes are used. Such colorants usually contain oxidation dye precursors, so-called developer components and coupler components, which form the actual dyes with one another under the influence of oxidizing agents—usually hydrogen peroxide. Oxidation dyes are exemplified by excellent, long-lasting dyeing results.

Oxidative color change agents usually come on the market in the form of two-component agents, in which two different preparations are packaged separately in two separate packages and are only mixed with one another shortly before use. The first component of common colorant kits is a water-containing composition—adjusted to be acidic for stability reasons—which contains dissolved hydrogen peroxide as oxidizing agent in concentrations of about 1.5 to about 24 wt. %. This aqueous oxidant composition is often in the form of an emulsion or dispersion and is usually provided in a plastic bottle with a resealable outlet (developer bottle).

This oxidizing agent formulation is mixed with a second preparation prior to use. This second preparation is an alkaline adjusted formulation, often in the form of a cream or gel, which also contains at least one oxidation dye precursor. This second preparation is often packaged in a tube or in a plastic or glass container.

In the usual form of application described above, the second preparation containing the alkalizing agent and the oxidation dye precursors is transferred from the tube or container to the developer bottle and then mixed by shaking with the hydrogen peroxide preparation already in the developer bottle. In this way, the application mixture is produced in the developer bottle. It is then applied to the hair via a small nozzle or outlet opening on the head of the developer bottle. The nozzle or outlet is opened after shaking and the application mixture can be removed by pressing the flexible developer bottle.

### BRIEF SUMMARY

Agents for dyeing hair and methods for using the same are provided. In an exemplary embodiment, an agent for dyeing hair includes a perborate, an oxidative dye precursor, a microbial gum, and an anionic cellulose. The agent also includes less than about 10% by weight of water.

A method for dyeing hair is provided in another embodiment. The method includes preparing a ready-to-use composition by mixing a composition comprising a perborate, an oxidative dye precursor, a microbial gum, and an anionic cellulose with water. The ready-to-use composition is then applied to the hair, and exposed to the hair for an exposure time. The ready-to-use composition is then rinsed from the hair.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

When preparing the application mixture in a bowl, both components—the first preparation containing the oxidizing agent and the second preparation containing alkalizing agent and, if desired, oxidation dye precursors—are transferred completely into a bowl or similar vessel and mixed there, for example with the aid of a brush. The application mixture is then removed from the mixing bowl using the brush. With this form of application, the use of a voluminous and expensive developer bottle is not necessary.

However, the filling of hydrogen peroxide solutions into such packaging is associated with problems, the cause of which lies in the reactivity of the hydrogen peroxide, which, depending on the storage conditions and possibly on the presence of decomposing impurities, can gradually decompose to form oxygen (i.e., gas).

The developer bottles known from the prior art are generally only filled to a maximum of half, usually only a third of their inner volume with the oxidizing agent composition. As a rule, developer bottles are made of polyethylene. Since polyethylene is permeable to both water vapor and gases, there is no or very little excess pressure in the developer bottle. In addition, developer bottles are usually provided with sturdy, thick walls and a stable screw cap, so that diffusion of the water vapor or gases is reduced by the thickness of the walls and a pressure increase within the bottle that occurs to a small extent has no negative effects.

As a result, the packaging is usually bulky, which compromises sustainability in terms of environmental protection and resource conservation. There would be an advantage if a solid were used as oxidant that could be activated by the addition of water alone, without the need to add further hydrogen peroxide. Sodium percarbonate, for example, is known as a solid oxidizing agent for dyeing preparations. One disadvantage of sodium percarbonate, however, is its reactivity to other recipe ingredients. Especially in formulations with a water content in the single-digit percentage range, sodium percarbonate can form undesirable reactions with other ingredients of the formulation under the influence of the water, which on the one hand leads to the consumption of the oxidizing agent and on the other hand also impairs the stability of the formulation. The coloring products available on the market are naturally stored for several weeks or months in the product packaging before they are applied by the user. For this reason, sodium percarbonate is not the oxidant of choice in a one-component colorant. Another group of solid oxidizing agents known from the prior art are the perborates. Perborates, such as sodium perborate, belong to the group of inorganic salts, just like percarbonates. A solid or powdery colorant with a high content of perborates therefore inevitably also has a high salt content.

Even solid or powdered hair dyes that are only mixed with water for application must contain at least one thickener to ensure sufficient viscosity of the ready-to-use product. However, many of the compounds commonly used as thickeners lose their thickening effect with increasing salt content. If the ready-to-use hair dye has too low a viscosity, it is more difficult to apply and can drip off the hair more easily during the application time, which is undesirable.

It has been the object of the present disclosure to provide a solid, powder or paste oxidative coloring agent which need only be mixed with water for the purpose of oxidative coloring. To enable the most resource-saving and sustainable packaging possible, the product should be available in the form of only one component, so that the separate portioning of different products in different packaging becomes superfluous. The agent should be stable and not have any disadvantages regarding its manageability, regarding the viscosity of the ready-to-use hair dye. In addition, the agent should also have good coloring properties and enable the oxidative coloring of keratin fibers with an intensive color result.

Surprisingly, it has now been shown that these tasks can be fully solved if a solid, powdery or paste-like coloring agent is applied to the keratin fibers, which contains at least one solid oxidizing agent from the group of perborates and at least one oxidation dye precursor, and which is further exemplified by its content of a special thickener combination of at least one microbial gum and at least one anionic cellulose. Due to its packaging in solid form, the water content is on average below 10% by weight.

A first object of the present disclosure is an agent for the oxidative dyeing of keratinous fibers, in particular human hair, comprising:

(a) at least one perborate,
(b) at least one oxidation dye precursor,
(c) at least one microbial gum,
(d) at least one anionic cellulose, and
(e) less than about 10% by weight of water, the percentage by weight being expressed in relation to the total weight of the product.

It has been found that in this way a one-component colorant can be made available which is stable in storage, and which quickly and reproducibly builds up the desired viscosity when the ready-to-use colorant is prepared by mixing with water. Surprisingly, the keratin fibers that were stained with this agent were also particularly intensely colored.

### Agent for the Oxidative Dyeing of Keratinous Fibers

Keratinic fibers, keratin containing fibers or keratin fibers are to be understood as furs, wool, feathers and in particular human hair. Although the agents as contemplated herein are primarily suitable for lightening and dyeing keratin fibers, in principle there is nothing to prevent their use in other areas.

The agents as contemplated herein are used for oxidative dyeing of keratinous fibers. The term “oxidative dyeing of keratin fibers” as used in the present disclosure includes any form of color change of the fibers in which at least one oxidation dye precursor and at least one oxidizing agent are used. This includes the color changes covered by the terms lightening, dyeing, oxidative dyeing, semi-permanent dyeing, permanent dyeing and nuancing. Explicitly included in the present disclosure are color changes that have a lighter color result compared to the original color, such as coloring bleaching.

The composition as contemplated herein contains the constituents (a), (b), (c) and (d) essential to the present disclosure and optionally the water components (e) present, preferably in a cosmetic carrier.

### Perborate

As the first constituent (a) essential to the present disclosure, the compositions as contemplated herein contain at least one perborate. Perborates are borates in which an Oxygen atom is replaced by a dioxygen group. Perborates as contemplated herein are inorganic compounds. A particularly suitable perborate is sodium perborate.

Sodium perborate is alternatively called sodium peroxoborate. The sodium perborate as contemplated herein is the commercially available sodium peroxoborate tetrahydrate (sodium perborate tetrahydrate) with the empirical formula (NaBO₃.4H₂O). Alternatively, the empirical formula NaBO₂.H₂O₂.3H₂O can be found in the literature. In the solid state, ring-shaped peroxoborates are present, with the formula Na₂B₂(O₂)₂(OH)₄.6H₂O. Sodium perborate tetrahydrate has the CAS No. 10486-00-7 and is commercially distributed, for example, by the company Sigma Aldrich®.

To achieve optimum color results, the perborate(s) is/are preferably used in certain ranges of amounts in the composition as contemplated herein. Particularly good results were obtained when the agent contains—based on its total weight—about 0.5 to about 14.0% by weight, preferably about 1.0 to about 12.0% by weight, more preferably about 1.2 to about 10.0% by weight, still more preferably about 1.4 to about 8.0% by weight and very particularly preferably about 2.0 to about 5.0% by weight of sodium perborate (b).

Within the framework of a particularly preferred embodiment, a technique as contemplated herein is exemplified by the fact that—in relation to its total weight—the agent contains

(a) about 0.5 to about 14.0% by weight, preferably about 1.0 to about 12.0% by weight, more preferably about 1.2 to about 10.0% by weight, still more preferably about 1.4 to about 8.0% by weight and most preferably about 2.0 to about 5.0% by weight of sodium perborate.

### Oxidation Dye Precursors

For coloring, the compositions as contemplated herein contain at least one oxidation dye precursor (b) as a second constituent essential to the present disclosure.

Oxidation dye precursors are classified into developer-type compounds and coupler-type compounds. In a preferred embodiment, the composition as contemplated herein comprises as oxidation dye precursor at least one developer component and optionally at least one coupler component. The developer components can form the actual dyes with each other, but preferably with coupler components. Preferably, therefore, the coloring compositions as contemplated herein comprise at least one oxidation dye precursor of the developer type and at least one oxidation dye precursor of the coupler type. The developer and coupler components can in principle be used in free form. In the case of substances with amino groups, however, it is preferable to use them in salt form, in the form of the hydrochlorides and hydrobromides or the sulfates.

Developer components and coupler components are generally used in approximately molar amounts to each other. Although molar use has been shown to be appropriate, a certain excess of individual oxidation dye precursors is not detrimental, so that developer components and coupler components in a molar ratio of about 3:1 to about 1:3, especially about 2:1 to about 1:1, may be included.

Suitable developer-type oxidation dye precursors are p-phenylenediamine and its derivatives. Preferred p-phenylenediamines are selected from one or more compounds of the group formed by p-phenylenediamine, p-tolylenediamine, 2-chloro-p-phenylenediamine, 2,3-dimethyl-p-phenylenediamine, 2,6-dimethyl-p-phenylenediamine, N,N-bis-(2-hydroxyethyl)-p-phenylenediamine, 2-(2-hydroxyethyl)-p-phenylenediamine, 2-(1,2-dihydroxyethyl)-p-phenylenediamine, N-(2-hydroxypropyl)-p-phenylenediamine, N-(4′-aminophenyl)-p-phenylenediamine, 2-methoxymethyl-p-phenylenediamine, N-phenyl-p-phenylenediamine, 2-(2-hydroxyethyloxy)-p-phenylenediamine and N-(4-amino-3-methyl-phenyl)-N-[3-(1H-imidazol-1-yl)propyl]amine as well as their physiologically tolerated salts.

As contemplated herein, it may further be preferred to use compounds containing at least two aromatic nuclei substituted with amino and/or hydroxyl groups as the developer component. Preferred dinuclear developer components are selected from N,N′-bis-(2-hydroxyethyl)-N,N′-bis-(4′-aminophenyl)-1,3-diaminopropan-2-ol, N,N′-bis-(4-aminophenyl)-1,4-diazacycloheptane and bis-(2-hydroxy-5-aminophenyl)methane and their physiologically acceptable salts.

Furthermore, it may be preferred as contemplated herein to use a p-aminophenol derivative or one of its physiologically compatible salts as the developer component. Preferred p-aminophenols are p-aminophenol, N-methyl-p-aminophenol, 4-amino-3-methylphenol, 4-amino-2-aminomethylphenol, 4-amino-2-(1,2-dihydroxyethyl)phenol and 4-amino-2-(diethylaminomethyl)phenol as well as their physiologically acceptable salts.

Furthermore, the developer component may be selected from o-aminophenol and its derivatives, preferably 2-amino-4-methylphenol, 2-amino-5-methylphenol, 2-amino-4-chlorophenol and/or their physiologically acceptable salts.

Furthermore, the developer component can be selected from heterocyclic developer components, such as pyrimidine derivatives, pyrazole derivatives, pyrazolopyrimidine derivatives or their physiologically compatible salts. Preferred pyrimidine derivatives are the compounds 2,4,5,6-tetraaminopyrimidine, 4-hydroxy-2,5,6-triaminopyrimidine and their physiologically tolerated salts. The preferred pyrazole derivative is 4,5-diamino-1-(2-hydroxyethyl)pyrazole and its physiologically tolerated salts. Pyrazolopyrimidines are particularly preferred as pyrazolo[1,5-a]pyrimidines.

Preferred developer-type oxidation dye precursors are selected from the group formed from p-phenylenediamine, p-toluenediamine, 2-(2-hydroxyethyl)-p-phenylenediamine, 2-(1,2-dihydroxyethyl)-p-phenylenediamine, N, N-bis-(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N′-bis-(2-hydroxyethyl)-N,N′-bis-(4-aminophenyl)-1,3-diamino-propan-2-ol, bis-(2-hydroxy-5-aminophenyl)methane, 1, 3-bis-(2,5-diaminophenoxy)-propan-2-ol, N,N′-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-amino-methylphenol, 4-amino-2-(1, 2-dihydroxyethyl)phenol and 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine or the physiologically tolerated salts of these compounds.

In a further embodiment of the first subject matter of the present disclosure, an agent as contemplated herein is exemplified in that it contains as oxidation dye precursor at least one developer component selected from the group of p-phenylenediamine, p-toluenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N′-bis-(2-hydroxyethyl)-N,N′-bis-(4-aminophenyl)-1,3-diamino-propan-2-ol, bis-(2-hydroxy-5-aminophenyl)methane, 1,3-bis-(2,5-diaminophenoxy)-propan-2-ol, N,N′-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-aminomethylphenol, 4-amino-2-(1,2-dihydroxy-ethyl)phenol and 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine or the physiologically tolerated salts of these compounds, and optionally additionally at least one coupler component selected from the group of 3-aminophenol, 5-amino-2-methylphenol 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline or the physiologically tolerated salts of the abovementioned compounds.

Very particularly suitable developer-type oxidation dye precursors are selected from at least one compound selected from the group of p-phenylenediamine, p-toluenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N′-bis-(2-hydroxyethyl)-N,N′-bis-(4-aminophenyl)-1,3-diamino-propan-2-ol, bis-(2-hydroxy-5-aminophenyl)methane, 1,3-bis-(2,5-diaminophenoxy)propan-2-ol, N,N′-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol, 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine, 2,3-diamino-6,7-dihydro-1H,5H-pyrazolo[1,2-a]pyrazol-1-one and physiologically tolerated salts thereof.

A further preferred embodiment of the present disclosure is exemplified in that the agent as contemplated herein further comprises, as oxidation dye precursor, in addition to at least one developer component, at least one coupler component. As a rule, m-phenylenediamine derivatives, naphthols, resorcinol and resorcinol derivatives, pyrazolones and m-aminophenol derivatives are used as coupler components.

Coupler components preferred as contemplated herein are

(A) m-Aminophenol and its derivatives, in particular 3-aminophenol, 5-amino-2-methylphenol, 3-amino-2-chloro-6-methylphenol, 5-amino-4-chloro-2-methylphenol, 5-(2′-hydroxyethyl)-amino-2-methylphenol and 2,4-dichloro-3-aminophenol,
(B) o-aminophenol and its derivatives, such as 2-amino-5-ethylphenol,
(C) m-Diaminobenzene and its derivatives such as, for example, 2,4-diaminophenoxy-ethanol, 1,3-bis-(2′,4′-diaminophenoxy)-propane, 1-methoxy-2-amino-4-(2′-hydroxyethylamino)benzene, 2,6-bis-(2′-hydroxyethylamino)-1-methylbenzene, 2-({3-[(2-hydroxyethyl)amino]-4-methoxy-5-methylphenyl}amino)ethanol and 2-({3-[(2-hydroxyethyl)amino]-2-methoxy-5-methylphenyl}amino)ethanol,
(D) o-Diaminobenzene and its derivatives,
(E) Di- or trihydroxybenzene derivatives, in particular resorcinol, 2-chlororesorcinol, 4-chlororesorcinol, 2-methylresorcinol and 1,2,4-trihydroxybenzene,
(F) Pyridine derivatives, 3-amino-2-methylamino-6-methoxypyridine, 2,6-diaminopyridine, 2,6-dihydroxy-3,4-dimethylpyridine, 2-amino-3-hydroxypyridine and 3,5-diamino-2,6-dimethoxy-pyridine,
(G) Naphthalene derivatives, such as 1-naphthol and 2-methyl-1-naphthol,
(H) Morpholine derivatives, such as 6-hydroxybenzomorpholine,
(I) Quinoxaline derivatives,
(J) Pyrazole derivatives, such as 1-phenyl-3-methylpyrazol-5-one,
(K) Indole derivatives, such as 6-hydroxyindole,
(L) Pyrimidine derivatives or
(M) Methylenedioxybenzene derivatives, such as 1-(2′-hydroxyethyl)-amino-3,4-methylenedioxybenzene, and their physiologically compatible salts.

Coupler components preferred as contemplated herein are selected from the group formed by 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2-(2, 4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4, 5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2, 6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline or the physiologically tolerated salts of the above compounds.

Particularly preferred coupler components as contemplated herein are resorcinol, 2-methylresorcinol, 5-amino-2-methylphenol, 3-aminophenol, 2-(2,4-diaminophenoxy)ethanol, 1,3-bis-(2,4-diaminophenoxy)propane, 1-methoxy-2-amino-4-(2′-hydroxyethylamino)benzene, 2-amino-3-hydroxypyridine, 2,6-dihydroxy-3,4-dimethylpyridine, 1,5-dihydroxynaphthalene, 2,7-dihydroxynaphthalene and 1-naphthol and a physiologically tolerated salt thereof.

To be able to provide a sustainable and environmentally friendly product, as little packaging material as possible should be used in the packaging of the agents as contemplated herein. For this reason, the agents as contemplated herein are preferably concentrates. Accordingly, the composition preferably contains the oxidation dye precursor(s) in relatively high total amounts which, based on the total weight of the composition, range from about 1.0 to about 60.0% by weight, preferably from about 1.5 to about 50.0% by weight, more preferably from about 1.7 to about 40.0% by weight, and most preferably from about 2.0 to about 35.0% by weight.

Within the framework of a particularly preferred embodiment, a technique as contemplated herein is exemplified in that it—in relation to its total weight—has the following properties

(b) the agent contains one or more oxidation dye precursors in a total amount of from about 1.0 to about 60.0% by weight, preferably from about 1.5 to about 50.0% by weight, more preferably from about 1.7 to about 40.0% by weight and most preferably from about 2.0 to about 35.0% by weight.

As already mentioned, the oxidation dye precursors—especially those of the developer type—represent reactive compounds that are first converted into their quinone or quinoneimine form under the action of the oxidizing agent and enter an oxidative coupling reaction with the couplers via this reactive intermediate stage, which leads to the final dye. To prevent a premature reaction and to increase the stability of the developers in the formulation that is not yet ready for use, the oxidation dye precursors are often used in the form of their salts.

Oxidation dye precursors of the developer type are usually derivatives of p-phenylenediamine, p-aminophenol or heterocyclic compounds with at least one, preferably at least two amino groups. To convert them into their salts, the amino groups contained in these structures are protonated and have the corresponding equivalents of sulphate anions, hydrogen sulphate anions, chloride anions and/or bromide anions to neutralize this positive charge. If the oxidation dye precursors are used in the form of their salts and the agent contains these in relatively high quantities, the salt load in the agent increases sharply. Agents with high salt content are particularly difficult to thicken, so finding suitable thickeners or thickener combinations is a major challenge. In the work leading to the present disclosure, it has now been surprisingly shown that the use of a special thickener combination of at least one microbial gum (c) and at least one anionic cellulose (d) makes it possible to achieve rapid, reproducible and controlled thickening even in the case of agents with a very high salt load, when these agents are mixed with water to produce the ready-to-use agent.

### Microbial Gum

For this reason, the composition as contemplated herein contains at least one microbial gum (c) as the third constituent (c) essential to the present disclosure.

For the purposes of the present disclosure, the term microbial gum means a substance produced by microorganisms during sugar fermentation. Examples of mircrobial gums or microbial gums include scleroglucan gums, gellane gums, pullulan gums, curdlan gums, xanthans, grifolan gums, lentinane gums, schizophyllan gums, spirulinan gums and crestin gums.

Particularly well-suited microbial gums can be selected from the group of xanthan gum, scleroglucan gum, gellan gum, pullulan gum, curdlan gum, grifolan gum, lentinan gum, schizophyllan gum and crestin gum, especially preferably xanthan gum.

Within the scope of a particularly preferred embodiment, a technique as contemplated herein is exemplified in that the agent contains

(c) at least one microbial gum selected from the group of xanthan gum, scleroglucan gum, gellan gum, pullulan gum, curdlan gum, grifolan gum, lentinan gum, schizophyllan gum and crestin gum, particularly preferably xanthan gum.

Xanthan gum can also be abbreviated as xanthan. Xanthan is produced with the help of bacteria of the genus Xanthomonas from sugar-containing substrates. The Backbone of the xanthan polymer is formed by β-(1→4)-linked D-glucose units. Attached to every other glucose unit is an α-(1→3)-glycosidic β-D-mannopyranosyl-(1→4)-β-D-glucuronopyranosyl-(1→2)-6-O-acetyl-α-D-mannopyranosyl side chain. Approximately half of the terminal mannose units of this side chain form Hydroxy groups at positions 4 and 6 to form a Ketal with Pyruvic acid. Occasionally, the acetyl group or a side chain may be completely missing. Xanthan bears the CAS number 11138-66-2.

Xanthan gum (alternatively also referred to as xanthan gum) is explicitly particularly suitable for use as a microbial gum in the compositions of the present disclosure.

Within the scope of an explicitly quite particularly preferred embodiment, a technique as contemplated herein is exemplified in that the agent
(c) contains xanthan gum.

Xanthan gum is a polyelectrolyte due to the presence of carboxylate groups. In the context of the present disclosure, the use of xanthans, at least about 95 wt. % of which have a particle diameter of less than about 0.180 mm mesh size in powder form and whose 1 wt. % solution in an aqueous 1 wt. % KCl solution (potassium chloride) has a viscosity of about 1400-1600 mPa s (measured with Brookfield® LVTD, spindle 3, 60 rpm, 25° C.), has been shown to be particularly advantageous. Such xanthans are commercially available, for example, under the trade name Keltrol® CG-SFT from the company CP Kelco®.

Scleroglucan gum or scleroglucan for short is a neutral exopolysaccharide formed by microorganisms (e.g., Sclerotium glucanicum) Exopolysaccharide (β-1,3;β-1,6-d-Glucan). Scleroglucan has the CAS number 39464-87-4.

Gellan gum, which can also be referred to simply as gellan, is an unbranched anionic microbial heteroexopolysaccharide with a basic tetrasaccharidic unit including the monomer's glucose, glucuronic acid and rhamnose. Approximately every basic unit is esterified with an L-glycerate and every second basic unit with an acetate. Gellan carries the CAS number 71010-52-1.

Pullulan gum, or pullulan for short, is a natural, water-soluble linear polysaccharide which is derived from Maltotriose-Units. Three Glucose-Units of maltotriose are represented by α-1,4-glycosidic linkages while successive maltotriose units are linked by α-1,6 compounds. Pullulan is produced with the help of the Fungus Aureobasidium pullulans from Starch and Sugar produced. Pullulan has the CAS number 9057-02-7.

Curdlan gum or Curdlan for short is a Homoglycan of glucose with glycosidic bonds of the β-(1,3) type, which is produced, among others, by Agrobacterium biobar, Euglena gracilis and Alcaligenes faecalis var. myxogenes. In some species the Periplasm Paramylons with additional β-(1,6)-bonds are present in the periplasm. Curdlan carries the CAS number 51052-65-4.

Grifolan gum or grifolan for short is a (1→3;1→6)-β-d-glucan from Grifola frondosa (Basidiomycota, Agariomycetes). Grifolan is a scleroglucan related to the (1→3;1→6)-β-d-Glucan.

Lentinan gum, or lentinan for short, is produced by the microbes from Lentinus edodes (sawfly, shiitake). Lentinan is a (1→3;1→6)-β-d-glucanrelated to scleroglucan. Lentinan is a compound derived from the Shiitake-Mushroom(Lentinula edodes). Chemically speaking, it is a Glucan, with each five straight-chain β-1,3-glycosidic linked Monomers there are two β-1,6-glycosidic branches. Lentinan carries the CAS number 37339-90-5.

Schizophyllan gum or schizophyllan for short is a(1→3;1→6)-β-d-glucan from Schizophyllum commune (Basidiomycota, Agariomycetes). Schizophyllan is a (1→3;1→6)-β-d-glucanrelated to scleroglucan.

Krestin gum or Kresti for shortn is a β-glucan protein complex (proteoglycan) from the white rot fungus Coriolus versicolor CM-101 (Syn. Trametes versicolor, Polyporus versicolor; Basidiomycota). It is obtained by extracting the mycelium of submerged cultures with hot water and precipitating with saturated ammonium sulphate solution.

To produce the optimum thickening effects, the microbial gum or gums are preferably used in specific ranges of amounts in the composition as contemplated herein. Rapid, reproducible and uniform thickening, even at high salt contents, could be achieved if the agent contained—based on its total weight—one or more microbial gums (c) in a total amount of from about 1.5 to about 12.0% by weight, preferably from about 2.0 to about 10.0% by weight, more preferably from about 3.0 to about 8.0% by weight and very preferably from about 4.5 to about 6.5% by weight.

Within the framework of a particularly preferred embodiment, a technique as contemplated herein is exemplified in that it—in relation to its total weight—has the following properties

(c) the agent contains one or more microbial gums in a total amount of from about 1.5 to about 12.0% by weight, preferably from about 2.0 to about 10.0% by weight, more preferably from about 3.0 to about 8.0% by weight, and most preferably from about 4.5 to about 6.5% by weight.

Within the framework of an explicitly quite particularly preferred embodiment, a technique as contemplated herein is exemplified in that it has—in relation to its total weight—the following features

(c) the agent contains about 1.5 to about 12.0% by weight, preferably from about 2.0 to about 10.0% by weight, more preferably from about 3.0 to about 8.0% by weight and most preferably from about 4.5 to about 6.5% by weight of xanthan gum.

### Anionic Celluloses (d)

Although some basic thickening can be achieved with the use of at least one mircobial gum (c), it has been found that the microbial gum (c) is not sufficient as the sole thickener in the compositions of the present disclosure. The work leading to the present disclosure has shown that optimum, rapid thickening can only be achieved if the compositions as contemplated herein contain at least one thickener (d) in addition to the thickener (c). For this reason, the composition as contemplated herein contains at least one anionic cellulose (d) as the fourth constituent essential to the present disclosure.

Cellulose is the main component of plant cell walls (about 50% by mass) and thus the most common organic compound and also the most common Polysaccharide. It is unbranched and includes several hundreds to tens of thousands of (β-1,4-glycosidically linked) β-D-Glucose- or Cellobiose-Units. Cellulose is a Polymer (polysaccharide ‘multiple sugar’) from the monomer cellobiose, which in turn is a Disaccharide (‘twofold sugar’) and Dimer of the Monosaccharide (‘simple sugar’) glucose. The monomers are linked together by β-1,4-glycosidic bonds. In the solid state, crystalline regions in cellulose alternate with those of low order (amorphous regions). Natural and manufacturing-related impurities, such as the presence of carboxy groups, are typically in the range of approx. 1%. As contemplated herein, cellulose itself is therefore not considered an anionic polysaccharide.

An anionic cellulose is understood to be a derivative of cellulose that carries at least one negative charge. This negative charge can come about, for example, through a deprotonated carboxyl group —COO—, a deprotonated sulfonyl group —SO₃— or a deprotonated phosphonate group —P(O)O₂²—. To maintain charge neutrality, these anionic groups are balanced by the presence of the appropriate amounts of cationic counterions such as Na+ ions, K⁺ ions or ammonium ion NH₄)⁺. Examples of carboxylate-containing monomers are carboxyalkyl ethers of sugars and sugar acids (uronic acids).

Anionic celluloses preferred as contemplated herein are carboxy-C₁-C₆-alkyl celluloses, which are preferably used in the form of their physiologically compatible salts. Physiologically compatible salts are the ammonium salts as well as the salts of alkali metals, in particular sodium and potassium, alkaline earth metals, in particular magnesium and calcium, as well as of zinc.

Carboxy-C₁-C₆-alkyl celluloses are Cellulose ethers, i.e. Derivatives of Cellulose which are part of the Hydroxy groups as Ether with a Carboxy-C₁-C₆-alkyl group (—CH₂)ₙ—COOH) linked. The index number n here indicates the number of —CH₂ groups, which can range from 1 to 6.

As a very particularly preferred anionic polysaccharide, carboxymethylcellulose is used either in free form or in the form of its physiologically compatible salt, especially its sodium salt. In its acid form, carboxymethyl cellulose carries the CAS number 9000-11-7. The sodium salt of carboxymethyl cellulose has the CAS number 9004-32-4.

Within the scope of a particularly preferred embodiment, a technique as contemplated herein is exemplified in that the agent

(d) contains at least one anionic cellulose from the group of carboxy-C₁-C₆-alkyl celluloses and their physiologically acceptable salts, particularly preferably from carboxymethyl cellulose and its physiologically acceptable salts.

Within the scope of a particularly preferred embodiment, a technique as contemplated herein is exemplified in that the agent

(d) contains sodium carboxymethyl cellulose.

The thickener sodium carboxymethylcellulose is commercially available under the trade name “Cekol® 50000”, for example. Sodium carboxymethyl cellulose (CMC) is also available for purchase from Sunray®. Another supplier of carboxymethyl cellulose is the company Ashland®, which markets this raw material under the trade name Blanose® CMC 9H4F.

To achieve the optimum synergistic effects in combination with the thickening agent (c), the aninionic celluloses (d) are also preferably used in certain quantity ranges in the composition as contemplated herein. A total content of anionic celluloses in the composition as contemplated herein which—based on the total weight of the composition—is in the range from about 10.0 to about 28.0% by weight, preferably from about 12.0 to about 26.0% by weight, more preferably from about 14.0 to about 24.0% by weight, still more preferably from about 16.0 to about 22.0% by weight and very particularly preferably from about 17.0 to about 21.0% by weight has been found to be particularly suitable for solving the problem as contemplated herein.

Within the framework of a particularly preferred embodiment, a technique as contemplated herein is exemplified in that it—in relation to its total weight—has the following properties

(d) the agent contains one or more anionic celluloses in a total amount of from about 10.0 to about 28.0% by weight, preferably from about 12.0 to about 26.0% by weight, more preferably from about 14.0 to about 24.0% by weight, still more preferably from about 16.0 to about 22.0% by weight, and most preferably from about 17.0 to about 21.0% by weight.

Within the framework of a particularly preferred embodiment, a technique as contemplated herein is exemplified in that it—in relation to its total weight—has the following properties

(d) the agent includes about 10.0 to about 28.0% by weight, preferably from about 12.0 to about 26.0% by weight, more preferably from about 14.0 to about 24.0% by weight, still more preferably from about 16.0 to about 22.0% by weight, and most preferably from about 17.0 to about 21.0% by weight of sodium carboxymethylcellulose.

In summary, very particularly preferred is an agent for the oxidative coloring of keratinous fibers, in particular human hair, containing—based on its total weight—

(a) about 0.5 to about 14.0% by weight of sodium perborate,
(b) one or more oxidation dye precursors in a total amount of about 1.0 to about 60.0% by weight
(c) about 1.5 to about 12.0% by weight xanthan gum,
(d) about 10.0 to about 28.0% by weight of sodium carboxymethyl cellulose, and
(e) less than about 10% by weight of water.

Very particularly preferred is also an agent for the oxidative coloring of keratinous fibers, in particular human hair, containing—based on its total weight—

(a) about 1.0 to about 12.0% by weight, sodium perborate,
(b) one or more oxidation dye precursors in a total amount of from about 1.0 to about 60.0% by weight,
(c) about 2.0 to about 10.0% by weight xanthan gum,
(d) about 12.0 to about 26.0% by weight of sodium carboxymethyl cellulose, and
(e) less than about 10% by weight of water.

Very particularly preferred is also an agent for the oxidative coloring of keratinous fibers, in particular human hair, containing—based on its total weight—

(a) about 1.2 to about 10.0% by weight sodium perborate,
(b) one or more oxidation dye precursors in a total amount of from about 1.0 to about 60.0% by weight,
(c) about 3.0 to about 8.0% by weight xanthan gum,
(d) about 14.0 to about 24.0% by weight of sodium carboxymethyl cellulose, and
(e) less than about 10% by weight of water.

Very particularly preferred is also an agent for the oxidative coloring of keratinous fibers, in particular human hair, containing—based on its total weight—

(a) about 2.0 to about 5.0 wt. % Sodium perborate,
(b) one or more oxidation dye precursors in a total amount of from about 1.0 to about 60.0% by weight,
(c) about 4.5 to about 6.5% by weight xanthan gum,
(d) about 17.0 to about 21.0% by weight of sodium carboxymethyl cellulose, and
(e) less than about 10% by weight of water.

### Water Content of the Agent

The agents as contemplated herein are available in the form of a one-component agent, which only needs to be mixed with water to produce the ready-to-use agent. Mixing with a second, separately packaged preparation can be omitted in this way, and packaging material and the associated costs can be saved. When mixed with water, hydrogen peroxide (or “active oxygen”) is released from the perborates in situ. Since contact with water converts the agent into its ready-to-use form, the agent itself is essentially anhydrous and thus contains less than about 10.0% by weight of water (e). For example, 100 g of an agent as contemplated herein contain at most about 9.9% by weight (=9.9 g) of water.

However, various raw materials may contain small amounts of water, for example if the raw materials are used in the form of an emulsion, contain water of crystallization, or water is present as a minor component that is difficult to avoid. Therefore, when using these raw materials, smaller amounts of water can be introduced into the compositions as contemplated herein. Up to a water content of just under about 10 wt. %, a good dyeing performance of the agents could be guaranteed. However, it has been found preferable to choose a lower water content.

Particularly preferred is an agent for the oxidative dyeing of keratinous fibers containing—based on its total weight—(e) less than about 8.0% by weight of water, more preferably less than about 6.0% by weight of water, still more preferably less than about 4.0% by weight of water, still more preferably from about 0.1 to about 3.5% by weight of water and very particularly preferably from about 0.5 to about 3.0% by weight of water.

In a very particularly preferred embodiment, a composition as contemplated herein is exemplified in that it—based on its total weight—comprises

(e) less than about 8.0% by weight, preferably less than about 6.0% by weight, more preferably less than about 4.0% by weight, still more preferably from about 0.1 to about 3.5% by weight and most preferably from about 0.5 to about 3.0% by weight of water.

The water content of an agent can be determined mathematically by adding up the amounts of water present in the individual ingredients. Furthermore, the water content of an agent can also be measured with a moisture analyzer or moisture meter, for example with a moisture meter from Mettler®, model Mettler® HS 153, whereby the loss on drying is determined at about 105° C., shut-off criterion 50 seconds with a product weight of about 1 to about 1.5 grams.

A very manageable and convenient form of low-water or essentially water-free packaging for the user is the provision of the agent in the form of a powder or a paste. Very preferably, therefore, the agent is in the form of a powder (such as a coloring powder) or in the form of a paste (such as a coloring paste).

In a particularly preferred embodiment, an agent as contemplated herein is exemplified in that it is in the form of a powder or in the form of a paste, particularly preferably in the form of a powder.

In a very particularly preferred embodiment, an agent as contemplated herein is exemplified in that it is in the form of a coloring powder.

In the context of the present disclosure, the terms “powder” or “powdery” are understood to mean those agents which include comminuted solid constituents, wherein the comminution may be achieved by trituration, crushing, grinding or by atomization-drying or freeze-drying. Thus, a powder is a mixture of small, solid particles. Powders can be composed of solid components with different particle sizes. Typically, however, it may be preferred if the powders have a particle size that is as homogeneous as possible, particularly to facilitate uniform dispersion or dissolution of the powders in water. A preferred powder in the sense of the present disclosure has an average particle diameter of at least about 20 μm and a BET surface area of about 40 to about 400 m²/g.

As contemplated herein, the terms “paste” or “pasty” are to be understood to mean a dosage form which has a viscosity at about 20° C. and 1013 mbar in the range from about 200,000 to about 1,600,000 mPas, preferably about 250,000 to about 1,400,000 mPas, particularly preferably about 300,000 to about 1,000,000 mPas, exceptionally preferably about 400,000 to about 750,000 mPas. The determination of paste viscosity is preferably carried out by employing Brookfield®; device RVDV II+; spindle no. 96, 4 revolutions per minute, at 20° C. Unless otherwise stated, all temperature data refer to a pressure of 1013 mbar.

### Weight Ratios (c) to (d)

In the development of the agents as contemplated herein, their targeted, rapid and reproducible thickening has proved to be a major challenge. It was found that optimal thickening can be solved by using the special thickener combination of microbial gum (c) and anionic cellulose (d). In this case, the thickening worked so well that the salt content of the agents—either by using the oxidation dye precursors in their salt form or by using other salt-like fillers—could also be chosen to be particularly high. The desired viscosity of the ready-to-use agent could be adjusted particularly well if certain ratios were selected when incorporating the thickeners (c) and (d). Thus, it was of advantage if the weight ratio of all the anionic celluloses (d) contained in the composition to all the microbial gums (c) contained in the composition, i.e., the weight ratio (d)/(c), was from about 10.0 to about 1.0, preferably from about 9.0 to about 1.5, more preferably from about 8.0 to about 2.0, still more preferably from about 6.0 to about 2.5 and most preferably from about 5.0 to about 3.0.

In other words, it has been found to be particularly advantageous if the anionic celluloses (d), especially the sodium carboxymethyl cellulose, are used in an excess of about 1 to about 10 times the weight of the microbial gum (c), especially the xanthan gum. The best results were obtained when the anionic cellulose (d) was incorporated into the agent in a three- to fivefold excess by weight compared to the microbial gum (c).

In a particularly preferred embodiment, a composition as contemplated herein is exemplified in that the weight ratio of all anionic celluloses (d) contained in the composition to all microbial gums (c) contained in the composition, i.e. the weight ratio (d)/(c), is from about 10.0 to about 1.0, preferably from about 9.0 to about 1.5, even more preferably from about 8.0 to about 2.0. i.e. the weight ratio (d)/(c), is from about 10.0 to about 1.0, preferably from about 9.0 to about 1.5, more preferably from about 8.0 to about 2.0, still more preferably from about 6.0 to about 2.5 and very particularly preferably from about 5.0 to about 3.0.

Example: 100 g of a powdered coloring agent contains about 5.0 g of xanthan (c) and about 18.0 g of sodium carboxymethylcellulose. The weight ratio (d)/(c) is 3.6.

### Alkalizing Agent

The composition as contemplated herein is preferably such that the ready-to-use composition obtained by mixing with water has an alkaline pH. Preferably, the composition ready for use has a pH value of about 8 to about 11.5, particularly preferably a pH value of about 8.5 to about 11, exceptionally preferably a pH value of about 9.0 to about 10.5, in each case measured at 20° C.

It can therefore be advantageous to additionally incorporate a further alkalizing agent into the agents. Since the agents are preferably made up in the form of a powder or paste, particularly well-suited alkalizing agents are solid at room temperature (20° C.).

The alkalizing agents which can be used as contemplated herein are preferably selected from the group formed by (earth) alkali metal metasilicates, (earth) alkali metal metasilicates, (Earth) alkali metal hydroxides, (Earth) alkali metal phosphates, (Earth) alkali metal hydrogen phosphates and basic amino acids. Preferred alkali metal ions are lithium, sodium and/or potassium. The preferred alkaline earth metal ions are magnesium and/or calcium.

Particularly suitable basic amino acids are arginine, histidine and lysine and/or their salts. Among the salts of arginine, lysine and histidine which are preferably suitable as contemplated herein are the ammonium salts, alkali metal salts and alkaline earth metal salts, in particular the lithium, sodium, potassium, magnesium and calcium salts, also the hydro halides, the hydrochlorides, and mixtures of these salts. A particularly preferred amino acid salt as contemplated herein is lysine hydrochloride. The amino acids suitable as contemplated herein, selected from arginine, lysine, histidine and their salts, may also contain water of crystallization.

In a particularly preferred embodiment, an agent as contemplated herein is exemplified in that it contains one or more alkalizing agents selected from the group of (earth) alkali metal silicates, (earth) alkali metal metasilicates, (earth) alkali metal hydroxides, (earth) alkali metal phosphates and (earth) alkali metal hydrogen phosphates, (earth) alkali metal carbonates and basic amino acids.

The specialist selects the quantities of alkalizing agent(s) to be used depending on the pH value to be set in the ready-to-use agent.

Thus, the composition may contain—based on its total weight—one or more alkalizing agents selected from the group of (earth) alkali metal silicates, (earth) alkali metal metasilicates, (earth) alkali metal hydroxides, (earth) alkali metal phosphates and (earth) alkali metal hydrogen phosphates and basic amino acids in a total amount of about 5.0 to about 60.0 wt. %, preferably of about 10.0 to about 55.0 wt. %, further preferably of about 15.0 to about 50.0 wt. % and most preferably of about 20.0 to about 45.0 wt. %% by weight, preferably from about 10.0 to about 55.0% by weight, further preferably from about 15.0 to about 50.0% by weight and most preferably from about 20.0 to about 45.0% by weight.

### Fillers

The composition as contemplated herein contains the components (a), (b), (c) and (d) essential to the present disclosure—as well as the water component (e), if present—preferably in a cosmetic carrier. In the case of solid, powder or paste-like agents, this cosmetic carrier is preferably solid, powder or paste-like fillers. Furthermore, the agent may optionally also contain anti-caking agents and/or drying agents.

In a further preferred embodiment, the composition as contemplated herein therefore contains at least one excipient selected from fillers, anti-caking agents and drying agents, and mixtures of these excipients. These excipients are intended to prevent the powder components from clumping or caking. Particularly preferred fillers are selected from sodium sulphate and sodium chloride and mixtures thereof.

Particularly preferred anti-caking agents are selected from fumed silicas, precipitated silicas, diatomaceous earth, calcium phosphate, calcium silicates, aluminum oxide, magnesium oxide, magnesium carbonate, zinc oxide, stearates, and mixtures thereof.

Particularly preferred desiccants are selected from sodium sulphate, sodium carbonate, magnesium sulphate, calcium chloride, precipitated silicas and fumed silicas and mixtures thereof.

The use of sodium sulfate and silicon dioxide has proved to be particularly preferable.

Silicon dioxide can be used, for example, in the form of amorphous silica. This substance has the CAS numbers 7631-86-9 and 112926-00-8 and is commercially available under the trade name Sipernat® 22 “Amorphous silica” from Evonik®.

In another particularly preferred embodiment, an agent as contemplated herein is exemplified in that it contains at least one filler selected from the group of sodium sulfate and silicon dioxide.

In another particularly preferred embodiment, an agent as contemplated herein is exemplified in that it contains at least one filler selected from the group of sodium sulfate and silicon dioxide.

In another particularly preferred embodiment, an agent as contemplated herein is exemplified in that it contains the fillers sodium sulfate and silicon dioxide. The fillers are preferably used in certain quantity ranges in the medium.

In another particularly preferred embodiment, an agent as contemplated herein is exemplified in that it contains—based on its total weight—about 10.0 to about 65.0% by weight, preferably about 15.0 to about 65.0% by weight, further preferably about 20.0 to about 60.0% by weight and very particularly preferably about 25.0 to about 55.0% by weight of sodium sulfate.

In a further particularly preferred embodiment, an agent as contemplated herein is exemplified in that it contains—based on its total weight—about 2.0 to about 30.0% by weight, preferably about 2.0 to about 26.0% by weight, further preferably about 2.0 to about 20.0% by weight and very particularly preferably about 2.0 to about 12.0% by weight of silicon dioxide.

### Other Components

In addition to the ingredients mentioned so far, the compositions as contemplated herein may optionally contain further ingredients. Thus, the preparations—especially if they are to be made up as a paste—may additionally contain at least one oil. Preferred oils can be selected from paraffin oil, silicone oil or ester oil as well as mixtures of these oils.

Further oils preferred as contemplated herein are selected from natural and synthetic hydrocarbons, particularly preferably paraffin oils, C₁₈-C₃₀ isoparaffins, isoeicosane, polyisobutenes and polydecenes, further selected from C₅-C₁₆ isoparaffins, isodecane, isododecane, isotetradecane and isohexadecane, and mixtures thereof, and 1,3-di-(2-ethylhexyl)-cyclohexane.

Further oils preferred as contemplated herein are selected from the benzoic acid esters of linear or branched C8-22 alkanols. Particularly preferred are benzoic acid C12-C15 alkyl esters.

Other preferred oils as contemplated herein are selected from fatty alcohols with about 6-30 carbon atoms, which are unsaturated or branched and saturated or branched and unsaturated. Preferred alcohol oils are 2-hexyldecanol, 2-octyldodecanol, 2-ethylhexyl alcohol and isostearyl alcohol, and mixtures thereof.

Other cosmetic oils preferred as contemplated herein are selected from the triglycerides (=triple esters of glycerol) of linear or branched, saturated or unsaturated, optionally hydroxylated C8-30 fatty acids. The use of natural oils, e.g. Amaranth seed oil, apricot kernel oil, argan oil, avocado oil, babassu oil, cottonseed oil, borage seed oil, camelina oil, safflower oil, peanut oil, pomegranate seed oil, grapefruit seed oil, hemp oil, hazelnut oil, elderberry seed oil, olive oil, mandarin oil, jojoba seed oil, linseed oil, jojoba seed oil, linseed oil, jojoba seed oil, linseed oil, jojoba seed oil, linseed oil, jojoba oil, linseed oil Palm kernel oil, Brazil nut oil, pecan oil, peach kernel oil, rapeseed oil, castor oil, sea buckthorn pulp oil, sea buckthorn oil, sesame oil, soybean oil, sunflower oil, grape seed oil, walnut oil, wild rose oil, wheat germ oil, and the liquid fractions of coconut oil and the likes. However, synthetic triglyceride oils, especially capric/caprylic triglycerides, are also preferred.

Other cosmetic oils particularly preferred as contemplated herein are selected from the dicarboxylic acid esters of linear or branched C₂-C₁₀ alkanols, diisopropyl adipate, di-n-butyl adipate, di-(2-ethylhexyl)adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl)succinate.

Other cosmetic oils particularly preferred as contemplated herein are selected from the esters of linear or branched saturated or unsaturated fatty alcohols having 2-30 carbon atoms with linear or branched saturated or unsaturated fatty acids having 2-30 carbon atoms, which may be hydroxylated. These preferably include 2-hexyldecyl stearate, 2-hexyldecyl laurate, isodecyl neopentanoate, isononyl isononanoate, 2-ethylhexyl palmitate and 2-ethylhexyl stearate, Isopropyl myristate, isopropyl palmitate, isopropyl stearate, isopropyl isostearate, isopropyl oleate, isooctyl stearate, isononyl stearate, isocetyl stearate, isononyl isononanoate, Isotridecyl isonanoate, cetearyl isonanoate, 2-ethylhexyl laurate, 2-ethylhexyl isostearate, 2-ethylhexyl cocoate, 2-octyl dodecyl palmitate, butyloctanoic acid-2-butyloctanoate, diisotridecyl acetate, n-butyl stearate, n-hexyl laurate, n-decyl oleate, oleyl oleate, oleylerucate, erucyl oleate, erucyl erucate and ethylene glycol dioleate.

Further preferred cosmetic oils as contemplated herein are selected from the adducts of 1 to 5 propylene oxide units with mono- or polyvalent C₈₋₂₂-Alkanols, such as octanol, decanol, decanediol, lauryl alcohol, myristyl alcohol and stearyl alcohol, e.g., B. PPG-2 myristyl ether and PPG-3 myristyl ether. Further cosmetic oils preferred as contemplated herein are selected from the addition products of at least 6 ethylene oxide and/or propylene oxide units to mono- or polyvalent C₃₋₂₂ alkanols such as glycerol, butanol, butanediol, myristyl alcohol and stearyl alcohol, which, if desired, may be esterified, e.g., PPG-14 butyl ether, PPG-9 butyl ether, PPG-10 butanediol, PPG-15 stearyl ether and glycereth-7-diisononanoate.

Other cosmetic oils preferred as contemplated herein are selected from the C₈-C₂₂ fatty alcohol esters of monovalent or polyvalent C₂-C₇ hydroxycarboxylic acids, in particular the esters of glycolic acid, lactic acid, malic acid, tartaric acid, citric acid and salicylic acid, e.g., C₁₂-C₁₅ alkyl lactate.

Other cosmetic oils preferred as contemplated herein are selected from the symmetrical, asymmetrical or cyclic esters of carbonic acid with C₃₋₂₂ alkanols, C₃₋₂₂ alkanediols or C₃₋₂₂ alkanetriols, e.g., dicaprylyl carbonate, or the esters according to DE 19756454 A1, glycerol carbonate.

Other cosmetic oils suitable as contemplated herein are selected from the silicone oils, which include, for example, dialkyl and alkylaryl siloxanes, such as decamethylcyclopentasiloxane, dodecamethylcyclohexasiloxane, dimethylpolysiloxane and methylphenylpolysiloxane, but also hexamethyldisiloxane, octamethyltrisiloxane and decamethyltetrasiloxane. As contemplated herein, it may also be preferred to use mixtures of the above oils.

Furthermore, the compositions as contemplated herein may also contain anionic, nonionic and cationic zwitterionic and amphoteric surfactants.

All anionic surfactants suitable for use on the human body are suitable as anionic surfactants in the compositions as contemplated herein. These are exemplified by a water-solubilizing anionic group such as a carboxylate, sulphate, sulphonate or phosphate group and a lipophilic alkyl group with 8 to 30 C atoms. In addition, glycol or polyglycol ether groups, ester, ether and amide and hydroxyl groups may also be present in the molecule. Examples of suitable anionic surfactants are linear and branched fatty acids with 8 to 30 C atoms (soaps), alkyl ether carboxylic acids, acyl sarcosides, acyl taurides, acyl isethionates, sulphosuccinic acid mono, -dialkyl esters and sulphosuccinic acid monoalkyl polyoxyethyl esters, linear alkane sulfonates, linear alpha-olefin sulfonates, alkyl sulfates and alkyl ether sulfates, and alkyl and/or alkenyl phosphates. Preferred anionic surfactants are alkyl sulfates, alkyl ether sulfates and alkyl ether carboxylic acids, each having about 10 to about 18 carbon atoms, preferably about 12 to about 14 carbon atoms in the alkyl group, and up to about 12 glycol ether groups, preferably 2 to 6 glycol ether groups in the molecule. Examples of such surfactants are the compounds with the INCI designations sodium laureth sulphates, sodium lauryl sulphates, sodium myreth sulphates or sodium laureth carboxylates.

Zwitterionic surfactants are surface-active compounds which carry at least one quaternary ammonium group and at least one carboxylate, sulphonate or sulphate group in the molecule. Particularly suitable zwitterionic surfactants are the so-called betaines such as the N-alkyl-N,N-dimethylammonium glycinates, for example the cocoalkyl-dimethylammonium glycinate, N-acyl-aminopropyl-N,N-dimethylammonium glycinates, for example the cocoacylaminopropyl dimethylammonium glycinate, and 2-alkyl-3-carboxymethyl-3-hydroxyethyl imidazolines each having 8 to 18 carbon atoms in the alkyl or acyl group, and the cocoacylaminoethyl hydroxyethyl carboxymethyl glycinate. A preferred zwitterionic surfactant is the fatty acid amide derivative known under the INCI name cocamidopropyl betaine.

Amphoteric surfactants are surface-active compounds which, apart from a C₈-C₂₄ alkyl or acyl group, contain at least one free amino group and at least one —COOH or —SO₃H group in the molecule and can form internal salts. Examples of suitable amphoteric surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each with 8 to 24 C atoms in the alkyl group. Particularly preferred amphoteric surfactants are N-coconut alkyl aminopropionate, coconut acylaminoethyl aminopropionate and C₁₂-C₁₈ acyl sarcosine.

Nonionic surfactants contain e.g., a polyol group, a polyalkylene glycol ether group or a combination of polyol and polyglycol ether group as hydrophilic group. Such compounds are, for example, addition products of about 4 to about 50 mol ethylene oxide and/or 0 to about 5 mol propylene oxide to linear and branched fatty alcohols, to fatty acids and to alkylphenols, each with 8 to 20 C atoms in the alkyl group, ethoxylated mono-, di- and triglycerides, such as glycerol monolaurate+20 ethylene oxide, and glycerol monostearate+20 ethylene oxide, sorbitan fatty acid esters and adducts of ethylene oxide with sorbitan fatty acid esters such as polysorbates (Tween 20, Tween 21, Tween 60, Tween 61, Tween 81), adducts of ethylene oxide with fatty acid alkanolamides and fatty amines, and alkyl polyglycosides. Suitable nonionic surfactants are C8-C22 alkyl mono- and oligoglycosides and their ethoxylated analogues as well as ethylene oxide addition products to saturated or unsaturated linear fatty alcohols, each with about 2 to about 30 moles of ethylene oxide per mole of fatty alcohol.

Further oxidation compositions preferably used as contemplated herein are exemplified in that the at least one anionic surfactant is selected from alkyl sulphates, alkyl ether sulphates and alkyl ether carboxylic acids each containing about 10 to about 18 C atoms, preferably about 12 to about 14 C-atoms in the alkyl group and up to about 12 glycol ether groups, preferably 2 to 6 glycol ether groups, in the molecule.

In principle, all cationic surface-active substances suitable for use on the human body are suitable as cationic surfactants. These are exemplified by at least one water-solubilizing cationic group, such as a quaternary ammonium group, or by at least one water-solubilizing cationizable group, such as an amine group, and furthermore by at least one (lipophilically active) alkyl group having 6 to 30 C atoms or at least one (lipophilically active) imidazole group or at least one (lipophilically active) imidazylalkyl group.

Agents preferred as contemplated herein contain at least one cationic surfactant which is preferably selected from quaternary ammonium compounds having at least one C8-C24 alkyl radical, esterquats and amidoamines each having at least one C8-C24 acyl radical, and mixtures thereof. Preferred quaternary ammonium compounds having at least one C8-C24 alkyl radical are ammonium halides, in particular chlorides, and ammonium alkyl sulfates, such as methosulfates or ethosulfates, such as C8-C24 alkyltrimethylammonium chlorides, C8-C24 dialkyldimethylammonium chlorides and C8-C24 trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, as well as the imidazolium compounds known under the INCI designation quaternium-27, quaternium-83, quaternium-87 and quaternium-91. The alkyl chains of the surfactants mentioned above preferably have about 8 to about 24 carbon atoms.

Esterquats are cationic surfactants that contain both at least one ester function and at least one quaternary ammonium group as a structural element and furthermore at least one C8-C24 alkyl radical or C8-C24 acyl radical. Preferred esterquats are quaternized ester salts of fatty acids with triethanolamine, quaternized ester salts of fatty acids with diethanolalkylamines and quaternized ester salts of fatty acids with 1.2-dihydroxypropyl dialkylamines. Such products are sold under the trademarks Stepantex®, Dehyquart® and Armocare®. N,N-bis(2-palmitoyloxyethyl)dimethylammonium chloride, distearoylethyl dimonium methosulfates and distearoylethyl hydroxyethylmonium methosulfates are preferred examples of such esterquats.

Alkylamidoamines are usually produced by amidation of natural or synthetic C8-C24 fatty acids and fatty acid sections with di-(C1-C3)alkylaminoamines. A compound from this substance group which is particularly suitable as contemplated herein is stearamidopropyldimethylamine.

In addition, the compositions as contemplated herein may additionally comprise at least one direct dye. These are dyes that are absorbed directly onto the hair and do not require an oxidative process to form the color.

For the matting of undesirable residual color impressions caused by melanin degradation products, especially in the reddish or bluish range, certain direct dyes of the complementary colors are particularly preferred. Direct dyes are usually nitrophenylenediamines, nitroaminophenols, azo dyes, anthraquinones or indophenols. Direct dyes are known as anionic, cationic and nonionic direct dyes. The direct dyes are each preferably used in an amount of about 0.001 to about 2% by weight, based on the weight of the blonding paste or the alkalizing composition (Alk).

Preferred anionic direct dyes are the compounds known by the international names or trade name Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52, Bromophenol Blue and Tetrabromophenol Blue. Preferred cationic direct dyes are cationic triphenylmethane dyes, for example Basic Blue 7, Basic Blue 26, Basic Violet 2 and Basic Violet 14, aromatic systems substituted with a quaternary nitrogen group, such as Basic Yellow 57, Basic Red 76, Basic Blue 99, Basic Brown 16 and Basic Brown 17, cationic anthraquinone dyes, such as HC Blue 16 (Bluequat B), and direct dyes containing a heterocycle having at least one quaternary nitrogen atom, in particular Basic Yellow 87, Basic Orange 31 and Basic Red 51. The cationic direct dyes sold under the trademark Arianor® are also preferred cationic direct dyes as contemplated herein. Nonionic nitro and quinone dyes and neutral azo dyes are particularly suitable as nonionic direct dyes. Preferred nonionic direct dyes are those known under the international designations or trade names of HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]-benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid, and 2-chloro-6-ethylamino-4-nitrophenol. As contemplated herein, a combination of tetrabromophenol blue and acid red 92 is particularly preferred.

### Procedure

The agents as contemplated herein are used in processes for coloring keratinous fibers, in particular human hair.

A second object of the present disclosure, therefore, is a method for oxidative coloring of hair, comprising the following steps in the order indicated.

(I) Preparation of a ready-to-use composition by mixing a composition as disclosed in detail in the description of the first subject matter of the present disclosure with water,
(II) Apply the ready-to-use product prepared in step (I) to the hair,

### (III) Exposure and

(IV) Rinse the product off the hair.

In one particularly suitable embodiment, for example, the agent of the first subject matter of the present disclosure may be provided in a container, bottle or can. Here, the dimensions of the container can be chosen so that the container is only partially filled and allows further filling with water. The amount of water that must be added to produce the ready-to-use agent can be specified, for example, by a marking on the wall of the container. The application mixture is prepared by adding the appropriate amount of water and shaking the container, for example.

In a further embodiment, the composition as contemplated herein may also be provided in a sachet, pouch, can or other storage container. To prepare the ready-to-use agent, the user now transfers the sachet (or similar) completely into a bowl, glass or vessel and then adds the recommended amount of water—while shaking or stirring. The mixing ratio of agent as contemplated herein to water may be, for example, in a ratio of about 1 to about 5 parts by weight of the agent (i.e. the coloring agent of the first subject matter of the present disclosure) to 10 parts by weight of water, preferably from about 1 to about 4 parts by weight of the agent to 10 parts by weight of water, more preferably from about 1 to about 3 parts by weight of the agent to 10 parts by weight of water and most preferably from about 1.4 to about 2.0 parts by weight of the agent to 10 parts by weight of water.

A very particularly preferred method is therefore exemplified by the (I) Preparing a ready-to-use composition by mixing the composition with water in a ratio of from about 1 to about 5 parts by weight of composition to 10 parts by weight of water, preferably from about 1 to about 4 parts by weight of composition to 10 parts by weight of water, more preferably from about 1 to about 3 parts by weight of composition to 10 parts by weight of water and most preferably from about 1.4 to about 2.0 parts by weight of composition to 10 parts by weight of water.

For example, when mixing about 1.4 to about 2.0 parts by weight of the agent to 10 parts by weight of water, about 1.4 g to about 2.0 g of the coloring agent as contemplated herein is mixed with 10 g of water (or about 14 to about 20 g of the coloring agent as contemplated herein with 100 g of water). The preparation of the ready-to-use agent can be done by mixing it with cold or warm water. To increase user comfort, the agent is preferably mixed with water that has a temperature of about 30 to about 45° C.

In step (II), the application of the ready-to-use agent prepared in step (I) to the hair can be done, for example, with a brush, an applicette or simply with the gloved hand.

Preferably, the exposure time in step (III) of the process as contemplated herein is about 5 to about 60 min, about 5 to about 50 min, more preferably about 10 to about 45 min. During the exposure time of the agents on the fiber, it may be advantageous to support the color change process by applying heat. A reaction phase at room temperature is also as contemplated herein. In particular, the temperature during the exposure time is between about 20° C. and about 40° C., especially between about 25° C. and about 38° C. The agents already give good treatment results at physiologically tolerable temperatures of below about 45° C.

At the end of the color change process, all components on the keratin fibers are rinsed out of the hair with water or a surfactant-comprising cleanser. Commercially available shampoo can be used as a cleaning agent, whereby the cleaning agent can be dispensed with, and the rinsing process can be carried out with tap water if the color changing agent has a higher surfactant content.

What has been said regarding the techniques as contemplated herein and those as contemplated herein also applies mutatis mutandis to the methods as contemplated herein.

### Examples

### 1. Formulations

The following formulations were prepared (unless otherwise stated, quantities correspond to % by weight).

| | For e.g.: | For e.g.: | For e.g.: | For e.g.: |
|---|---|---|---|---|
| | E1 | V1 | E2 | V2 |
| Sodium perborate | 2.00 | — | 4.00 | — |
| Sodium percarbonate | — | 2.00 | — | 4.00 |
| Sodium carbonate | 2.00 | 2.00 | — | — |
| basic magnesium carbonate (magnesium carbonate | 8.00 | 8.00 | 8.00 | 8.00 |
| hydroxide) heavy pharm. 400 g/l | | | | |
| Aeroperl ® 300/30 (INCI: Silica, fumed silica) | 0.30 | 0.30 | 0.30 | 0.30 |
| Succinic acid | 2.40 | 2.40 | 1.20 | 1.20 |
| p-Toluylenediamine sulphate | 1.45 | 1.45 | 1.45 | 1.45 |
| 4-chlororesorcinol | 0.35 | 0.35 | 0.35 | 0.35 |
| 2,7-Dihydroxynaphthalene | 0.20 | 0.20 | 0.20 | 0.20 |
| 2-methylresorcinol | 0.15 | 0.15 | 0.15 | 0.15 |
| Resorcinol | 0.15 | 0.15 | 0.15 | 0.15 |
| m-Aminophenol | 0.04 | 0.04 | 0.04 | 0.04 |
| Sodium sulphate | 17.10 | 17.10 | 17.10 | 17.10 |
| Sodium metasilicate anhydrous fine | 6.00 | 6.00 | 6.00 | 6.00 |
| Sodium carboxymethyl cellulose (INCI: Cellulose | 19.00 | 19.00 | 19.00 | 19.00 |
| gum; Cekol ® 50000) | | | | |
| Xanthan NaTrue ® | 5.50 | 5.50 | 5.50 | 5.50 |
| Paraffinum Liquidum | 2.50 | 2.50 | 2.50 | 2.50 |
| Sipernate ® 22, (INCI: Silica, precipitated silica) | 6.00 | 6.00 | 6.00 | 6.00 |
| Sodium sulphate | ad 100 | ad 100 | ad 100 | ad 100 |
| | For e.g.: | For e.g.: | For e.g.: | For e.g.: |
| | E3 | V3 | E4 | V4 |
| Sodium perborate | 2.00 | — | 4.00 | — |
| Sodium percarbonate | — | 2.00 | — | 4.00 |
| Sodium carbonate | 2.00 | 2.00 | — | — |
| basic magnesium carbonate (magnesium carbonate | 8.50 | 8.50 | 8.50 | 8.50 |
| hydroxide) heavy pharm. 400 g/l | | | | |
| Aeroperl ® 300/30 (INCI: Silica, fumed silica) | 0.40 | 0.40 | 0.40 | 0.40 |
| Succinic acid | 2.40 | 2.40 | 1.20 | 1.20 |
| p-Toluylenediamine sulphate | 3.78 | 3.78 | 3.78 | 3.78 |
| 4-chlororesorcinol | 0.83 | 0.83 | 0.83 | 0.83 |
| 2,7-Dihydroxynaphthalene | 0.47 | 0.47 | 0.47 | 0.47 |
| 2-methylresorcinol | 0.31 | 0.31 | 0.31 | 0.31 |
| Resorcinol | 0.38 | 0.38 | 0.38 | 0.38 |
| m-Aminophenol | 0.10 | 0.10 | 0.10 | 0.10 |
| Sodium sulphate | 17.10 | 17.10 | 17.10 | 17.10 |
| Sodium metasilicate anhydrous fine | 6.00 | 6.00 | 6.00 | 6.00 |
| Sodium carboxymethyl cellulose (INCI: Cellulose | 19.00 | 19.00 | 19.00 | 19.00 |
| gum; Cekol 50000) | | | | |
| Xanthan NaTrue ® | 5.50 | 5.50 | 5.50 | 5.50 |
| Paraffinum Liquidum | 2.50 | 2.50 | 2.50 | 2.50 |
| Sipernate ® 22, (INCI: Silica, precipitated silica) | 6.00 | 6.00 | 6.00 | 6.00 |
| Sodium sulphate | ad 100 | ad 100 | ad 100 | ad 100 |

The powdered dyes produced in this way were not stored but used immediately after their production.

### 2. Application

Hair strands from Kerling (natural white) were measured calorimetrically with a spectrophotometer from Datacolor® (500).

| | L | a | b | C |
|---|---|---|---|---|
| Zero strand Kerling Natural White | 79.08 | −0.18 | 18.56 | 18.56 |

With stirring, 2 g of color powder were mixed with 10 g of water (tap water or city water) (water temperature 38° C.). The dyes could be mixed quickly and without clumping. The application mixture remained low viscosity in the first seconds of mixing, so that mixing could be done easily and quickly. Then the viscosity increased, and the ready-to-use dye could be applied well and evenly to the hair strands.

The ready-to-use dye obtained in this way was applied to the hair strands and left to act for 30 minutes. After that, the hair strands were washed with a commercial shampoo and water and dried.

Afterwards, the hair strands were measured using a farmetric method. The color difference (DE value) was determined from the Lab values obtained during the measurements.

The ΔE value (or DE value) used to assess color intensity is derived from the L*a*b* colorimetric values as follows:

ΔE=[(Lᵢ−L₀)²+(aᵢ−a₀)²+(bᵢ−b₀)]^{1/2}

L_{σ}, a₀ and b₀: Colorimetric values before dyeing
Lᵢ, aᵢ and bᵢ: Colorimetric values after dyeing

The ΔE value indicates the color difference that exists between the untreated and the treated hair strand. The greater the dE value, the greater the color difference (i.e., the color gap) between the uncolored and the colored hair and the stronger the coloring power.

### 3. Dyeing Capacity

| ready-to-use dye with | | L | a | b | DL | EN | |
|---|---|---|---|---|---|---|---|
| E1 | 2 g perborate | 34.01 | 4.24 | 11.79 | 45.07 | 45.78 | E1 better |
| V1 | 2 g percarborate | 35.29 | 4.53 | 12.72 | 43.79 | 44.43 | than V1 |
| E2 | 4 g perborate | 33.83 | 4.65 | 11.48 | 45.24 | 46.05 | E2 better |
| V2 | 4 g percarbonate | 36.39 | 4.43 | 13.44 | 42.68 | 43.24 | than V2 |

The ready-to-use coloring agents obtained starting from the agents E1 and E2 as contemplated herein showed a greater color shift and thus a higher color intensity than the corresponding comparative formulations V1 and V2.

| ready-to-use dye with | | L | a | b | DL | EN | |
|---|---|---|---|---|---|---|---|
| E3 | 2 g perborate | 23.79 | 3.94 | 7.49 | −55.29 | 56.54 | E3 better |
| V3 | 2 g percarborate | 24.70 | 4.48 | 8.47 | −54.37 | 55.50 | than V3 |
| E4 | 4 g perborate | 22.57 | 4.17 | 7.03 | −56.50 | 57.83 | E4 better |
| V4 | 4 g percarbonate | 25.04 | 5.15 | 9.63 | −54.04 | 55.03 | than V4 |

The ready-to-use coloring agents obtained starting from the agents E3 and E4 as contemplated herein showed a greater color shift and thus a higher color intensity than the corresponding comparative formulations V3 and V4.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An agent for the oxidative dyeing of human hair, comprising:
(a) a perborate,
(b) an oxidation dye precursor,
(c) a microbial gum,
(d) an anionic cellulose, and
(e) less than about 10% by weight of water, the percentage by weight of the water being expressed in relation to a total weight of the agent.

2. The agent according to claim 1, wherein the agent comprises; with respect to the total weight of the agent,
(a) from about 0.5 to about 14.0% by weight of the perborate.

3. The agent according to claim 1, wherein the agent comprises, based on the total weight of the agent,
(b) the oxidation dye precursor in a total amount of from about 1.0 to about 60.0% by weight.

4. The agent according to claim 1, wherein:
(c) the microbial gum is selected from the group of xanthan gum, scleroglucan gum, gellan gum, pullulan gum, curdlan gum, grifolan gum, lentinan gum, schizophyllan gum, spirulinan gum, crestin gum, and combinations thereof.

5. (canceled)

6. The agent according to claim 1, wherein:
(d) the anionic cellulose is selected from the group of carboxy-C₁-C₆-alkyl celluloses, their physiologically acceptable salts, and combinations thereof.

7. The agent according to claim 1, wherein:
(d) the anionic cellulose is present in the agent in a total amount of from about 10.0 to about 28.0% by weight, based on the total weight of the agent.

8. The agent according to claim 1, wherein the agent comprises, based on the total weight of the agent,
(e) less than about 8.0% by weight of water.

9. The agent according to claim 1, wherein the agent is in the form of a powder or in the form of a paste.

10. The agent according claim 1, wherein a weight ratio of the anionic cellulose (d) included in the agent to the microbial gum (c) included in the agent, i.e., the weight ratio (d)/(c), is from about 10.0 to about 1.0.

11. The agent according to claim 1, wherein the agent comprises an alkalizing agent selected from the group of (earth) alkali metal silicates, (earth) alkali metal metasilicates, (earth) alkali metal hydroxides, (earth) alkali metal phosphates, (earth) alkali metal hydrogen phosphates, (earth) alkali metal carbonates, basic amino acids, and combinations thereof.

12. The agent according to claim 1, wherein the agent comprises a filler selected from the group of sodium sulfate, silicon dioxide, and combinations thereof.

13. The agent according to claim 1, wherein the agent comprises—based on the total weight of the agent—from about 10.0 to about 65.0% by weight of sodium sulfate.

14. The agent according to claim 1, wherein the agent comprises—based on the total weight of the agent, from about 2.0 to about 30.0% by weight of silicon dioxide.

15. A method for the oxidative coloring of hair, comprising the following steps in the order indicated
(I) preparing a ready-to-use composition by mixing a composition comprising (a) a perborate, (b) an oxidation dye precursor, (c) a microbial gum, (d) an anionic cellulose, and (e) less than about 10% by weight of water, the percentage by weight being expressed in relation to a total weight of the agent, with water,
(II) apply the ready-to-use composition prepared in step (I) to the hair,
(III) exposing the hair to the ready-to-use composition for an exposure time, and
(IV) rinsing the ready-to-use composition off the hair.

16. The method according to claim 15, wherein:
(I) Preparing the ready-to-use composition comprises mixing the composition with the water in a ratio of from about 1 to about 5 parts by weight of the composition to about 10 parts by weight of the water.

17. The agent according claim 1, wherein a weight ratio of the anionic cellulose (d) included in the agent to the microbial gum (c) included in the agent, i.e., the weight ratio (d)/(c), is from about 5.0 to about 3.0.

18. The agent according to claim 2, wherein the perborate comprises sodium perborate.

19. The agent according to claim 18, wherein the agent comprises from about 2.0 to about 5.0 percent by weight of the sodium perborate, based on the total weight of the agent.

20. The agent according to claim 1, wherein the agent comprises, based on the total weight of the agent, from about 0.5 to about 3.0% by weight of the water.
